(19) United States
(12) Patent Application Publication
(10) Pub. No.: US 2011/0144361 A1
(43) Pub. Date: Jun. 16, 2011

(54) EFFICIENT PROCESS FOR PRODUCING EPOXIDES BY OXIDATION OF OLEFINS IN THE HOMOGENEOUS GAS PHASE

(75) Inventor: Torsten Berndt, Leinefelde (DE)

(73) Assignee: Zylum Beteiligungsgesellschaft mbH & Co. Patente II KG, Schoenefeld/Waltersdorf (DE)

(21) Appl. No.: 12/674,064

(22) PCT Filed: Aug. 12, 2008
(86) PCT No.: PCT/EP08/60571

§ 371 Date: Feb. 18, 2010

(30) Foreign Application Priority Data
Aug. 20, 2007 (DE) 10 2007 039 874.5

(51) Int. Cl. C07D 301/02

(52) U.S. Cl. 549/518

(57) ABSTRACT

An economical one-step process is provided for the preparation of epoxides by oxidation of olefins in a homogeneous gas phase reaction, wherein the olefin is reacted in a flow reactor with a gas mixture of ozone and NO₂ and/or NO as oxidants without use of a catalyst, and wherein ozone and NO₂ and/or NO are mixed in a mixing chamber connected upstream to the flow reactor. The process is characterized in that the olefin in the reaction zone of the flow reactor is reacted at a reaction temperature of approximately 150° C. to approximately 450° C. and a pressure of 250 mbar to 10 bar with the gas mixture of the oxidant, that the carrier gas flow containing the olefin is heated in a preheating zone of the flow reactor to a temperature of 250° C. to 650° C., and that the gas mixture of the oxidant from the mixing chamber, having ambient temperature, is turbulently mixed with the olefin in the reaction zone of the flow reactor, so that the reaction temperature is reached during the mixing and the ratio of olefin-gas flow and gas flow of the oxidant is 5:1 to 1:1.

### CROSS-REFERENCE TO RELATED APPLICATIONS

This application is a Section 371 of International Application No. PCT/EP2008/060571, filed Aug. 12, 2008, which was published in the German language on Feb. 26, 2009, under International Publication No. WO 2009/024503 A1 and the disclosure of which is incorporated herein by reference.

### BACKGROUND OF THE INVENTION

The invention is directed to an economical one-step process for the preparation of epoxides by oxidation of olefins in a homogeneous gas phase reaction, wherein the olefin is reacted in a flow reactor with a gas mixture of ozone and NO₂ and/or NO as oxidant without use of a catalyst, and whereby ozone and NO₂ and/or NO are mixed in a mixing chamber connected upstream to the flow reactor, characterized in that the olefin in the reaction zone of the flow reactor is reacted at a reaction temperature of about 150° C. to about 450° C. and a pressure of 250 mbar to 10 bar with the gas mixture of the oxidant, whereby the carrier gas flow containing the olefin is heated in a preheating zone of the flow reactor to a temperature of 250° C. to 650° C., the gas mixture of the oxidant from the mixing chamber, having ambient temperature, is turbulently admixed to the olefin in the reaction zone of the flow reactor, so that the reaction temperature is reached during the mixing and the ratio of olefin/gas flow and gas flow of the oxidant is 5:1 to 1:1.

It is well known to produce epoxides via the oxidation of olefins in a homogeneous gas phase reaction by using a gas flow of ozone/NOₓ as oxidant and carrying out the reaction under mild reaction conditions without use of a catalyst. WO 02/20502 A1 describes in the examples the oxidation of propylene, trans-butylene and iso-butylene under pressures of 10 to 25 mbar and temperatures between 140-230° C. The selectivities achieved for the epoxide produced are between 68.9 and 96.9%.

In Ind. Eng. Chem. Res, 2005(44), p. 645-650 Berndt, T. and Böge, O. describe further investigations concerning the epoxidation of propylene and ethylene in the gas phase. Propylene oxide and ethylene oxide are epoxides of economic interest, since they serve as precursors for the production of polymers (polyester, polyurethane) or solvents (glycols). The investigations described in this publication show on the one hand that the selectivity for propylene oxide declines considerably from 89.1% to 56.6% with rising pressure from 25, 50, 100 and 200 mbar (temperature 300° C.) (see p. 646, left column, “Results and Discussion”). On the other hand, the investigations showed that the molar ratio of reacted propylene to the ozone employed (ozone usage Δ [C₃H₆]/[O₃]₀) also declines with rising pressure (see p. 648, Table 3).

For the technical realization of an efficient industrial process, however, pressures being far below atmospheric pressure, are not suitable, since they require an increased pump capacity, resulting in negative implications on the investment and energy costs. Despite high pressures, the selectivity for epoxide in an industrial process should be at least 80%, and in particular the molar ratio of reacted epoxide to ozone employed should be 1 if possible (i.e. an ozone usage of 100%), since ozone is expensive.

### BRIEF SUMMARY OF THE INVENTION

This object is achieved by a process for the preparation of epoxides by oxidation of olefins in a homogeneous gas phase reaction, wherein the olefin, added by the use of a carrier gas, is reacted in a flow reactor with a gas mixture of ozone and NO₂ and/or NO as oxidants without use of a catalyst, and wherein ozone and NO₂ and/or NO are mixed in a mixing chamber connected upstream to the flow reactor, characterized in that the olefin in the reaction zone of the flow reactor is reacted at a reaction temperature of about 150° C. to about 450° C. and a pressure of 250 mbar to 10 bar with the gas mixture of the oxidant, that the carrier gas flow containing the olefin is heated in a preheating zone of the flow reactor to a temperature of 250° C. to 650° C., and that the gas mixture of the oxidant from the mixing chamber, having ambient temperature, is turbulently admixed to the olefin in the reaction zone of the flow reactor, so that the reaction temperature is reached during the mixing and the ratio of olefin-gas flow and gas flow of the oxidant is 5:1 to 1:1.

It was surprisingly found that despite high pressures of from 250 mbar to 10 bar, in particular pressures of from 500 to 2000 mbar, preferably of more than 1000 mbar, in particular preferred at atmospheric pressure, molar ratios of generated epoxide to ozone employed are achieved, which are almost 1 and even higher than 1, if the above conditions are followed. Currently, the surprising finding that the olefin reacted exceeds the ozone employed is mechanistically unclear. When following the conditions mentioned above, good selectivities of more than 80% and in some cases more than 90% are achieved.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown. In the drawings:

FIG. 1 is a graphical presentation of the results of the epoxidation of amylene according to the process described in Example 1 below; and

FIG. 2 is a graphical presentation of the results of the epoxidation of tetramethyl ethylene (TME) according to the process described in Example 2 below.

### DETAILED DESCRIPTION OF THE INVENTION

According to the invention, the carrier gas stream containing the olefin is preheated to a temperature of from 250 to 650° C., which is higher than the actual reaction temperature. Preferably, the olefin gas stream is preheated to 400 to 550° C. This is carried out in the preheating zone of the flow reactor. The gas flow consisting of ozone and NO₂ and/or NO and, if applicable, the carrier gas, is mixed in the mixing chamber, and is turbulently admixed to the reaction zone of the flow reactor (preferably downstream at the beginning of the reaction zone) at ambient temperature (18 to 25° C.), so that (at least) the reaction temperature is reached immediately (“immediately” meaning that the reaction temperature is reached within the first 5 to 10% of the residence time in the reaction zone). The reaction temperature is about 150 to about 450° C., preferably from about 200 to about 350° C.

According to the present invention, the term “turbulent mixing” is to be understood for example as an insertion of the gas flow of oxidant via nozzles, via the use of a grid or by using a turbulent free jet or other suitable methods. In any case, an immediate, ideal mixing should be achieved.

According to the invention, the ratio of olefin-gas flow and the gas flow of the oxidant is chosen so that the reaction temperature is reached after the turbulent mixing. The ratio of olefin-gas flow to the gas flow of the oxidant is from 5:1 to 1:1, preferably from 4:1 to 2:1.

The residence time in the reaction zone is from 1 ms to several seconds at a maximum. One ms to 250 ms are preferred.

According to the invention, ozone is used preferably as ozone/oxygen mix, in particular having 1-15 vol. % ozone in the oxygen, preferably having 5-10 vol. % ozone in the oxygen. Ozone and NO₂ are used in a ratio below 0.5. Ozone and NO are preferably used in a ratio below 1.5.

The carrier gas for the olefin and for the gas mixture of oxidant can be an inert gas, such as helium, argon or nitrogen, air or oxygen or mixtures of the gases mentioned. Nitrogen is preferred.

The process according to the invention is carried out in a flow reactor, as in principle described in WO 02/20502 A1. The flow reactor according to the invention besides the reaction zone solely comprises a preheating zone for the preheating of the olefin gas flow, which extends to the beginning of the reaction zone and is directly connected to it without interruption and which is heated independently from the reaction zone.

The process according to the present invention allows the oxidation of any compound having olefinic double bonds in the molecules to epoxides. One, two or more olefinic double bonds can be contained per molecule. The olefinic compounds can also include hetero atoms such as oxygen, sulphur and/or nitrogen. The olefinic compounds can therefore be pure hydrocarbons, esters, alcohols, ethers, acids, amines, carbonyl compounds or polyfunctional compounds, preferably having 2 to 30 carbon atoms in the molecule, in particular at least 3 carbon atoms. The process can in particular be used for straight chain compounds, branched or cyclic compounds, substituted or unsubstituted aliphatic olefinic compounds or olefinic compounds having an aryl proportion in the molecule, in particular for olefinic compounds having 2 to 30 carbon atoms, preferably having at least 3 carbon atoms. Substituents containing halogen or oxygen, or sulphur or nitrogen can be used for the substituted olefinic compounds.

### EXAMPLES

### Example 1

### Epoxidation of amylene (2-methyl-2-butene) at 300° C. and 500 mbar at Different Feed-Ratios amylene/O₃ of 1.43 to 3.47

The olefin gas flow (4 standard-liter/min.) consisting of amylene and N₂ is preheated to 550° C. The O₃/NOₓ gas flow (2 standard-liter/min.) consisting of 6.5 vol. % NO₂, 36 vol. % of an O₃/O₂ mixture (from ozone generator) and 57.5 vol. % N₂, starting from room temperature, is brought into contact with the preheated olefin gas flow via nozzles. The reaction temperature is 300° C. After mixing, the ozone content is 0.7 vol. % and the amylene-content is 1.0 to 2.4 vol. %. The bulk-residence time in the reaction zone is 4.8 ms.

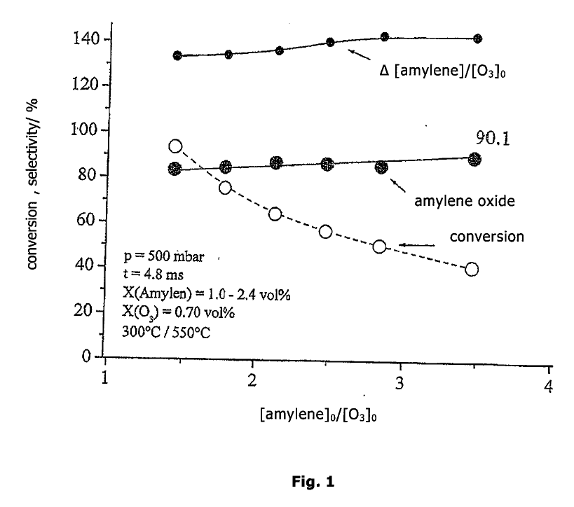

As side products, acetaldehyde and acetone are found. The results are presented in FIG. 1.

The parameters at the working point of highest selectivity at the feed ratio amylene/O₃=3.47 are:
conversion of amylene: 41.3%
selectivity for amylene oxide: 90.1 mol. %
reacted amylene/O₃ employed: 1.43 (molar)
space-time-yield: 6240 g amylene oxide/hr/(liter reactor volume).

### Example 2

### Epoxidation of TME (tetramethyl ethylene) at 200° C. and 500 mbar with Different Feed-Ratios TME/O₃ of 1.43 to 5.24

The olefin gas flow (2 standard liter/min.) consisting of TME and N₂ is preheated to 320° C. The O₃/NOₓ gas flow (1 standard liter/min.) consisting of 6 vol. % NO₂, 25 vol. % of an O₃/O₂ mixture (from ozone generator) and 69 vol. % N₂, starting from room temperature, is brought into contact via nozzles to the preheated olefin gas flow. The reaction temperature is 200° C. After admixture, the ozone content is 0.59 vol. % and the TME content 0.84-3.1 vol. %. The bulk residence time in the reaction zone is 9.6 ms.

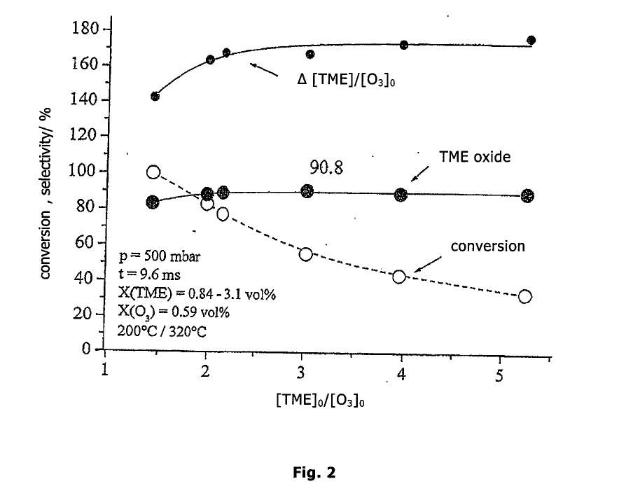

Acetone and pinacolone (trimethyl acetone) are found as side products. The results are presented in FIG. 2.

The parameters at the working point of highest selectivity at the feed ratio TME/O₃=3.02 are:
TME conversion: 55.6%
selectivity for TME oxide: 90.8 mol. %
converted TME/O₃ employed: 1.68 (molar)
space-time-yield: 3600 g TME oxide/hr/(liter reactor volume).

### Example 3

### Epoxidation of propylene at 300° C. and 500 mbar

The olefin gas flow (4 standard liter/min.) consisting of propylene and N₂ is preheated to 550° C. The O₃/NOₓ gas flow (2 standard liter/min.) consisting of 2.25 vol. % NO₂, 10 vol. % of an O₃/O₂ mixture (from ozone generator) and 87.75 vol. % N₂, starting from room temperature, is brought into contact via nozzles to the preheated olefin gas flow. The reaction temperature is 300° C. After admixture, the ozone content is 0.27 vol. % and the propylene content 5.6 vol. %. The bulk-residence time in the reaction zone is 4.8 ms.

Formaldehyde and acetaldehyde are found as side products.

The parameters at the working point are:
conversion of propylene: 4.6%
selectivity for propylene oxide: 81.3 mol. %
propylene conversion/O₃ employed: 0.98 (molar)
space-time-yield: 980 g propylene oxide/hr/(liter reactor volume).

### Example 4

### Epoxidation of TME (tetra methyl ethylene) at 300° C. and 1000 mbar at Different O₂-Contents in the Reaction Gas

The olefin gas flow (4 standard liter/min.) consisting of TME and N₂ is preheated to 460° C. The O₃/NOₓ gas flow (2 standard liter/min.) consisting of 5 vol. % NO₂ and 25 vol. % of an O₃/O₂ mixture (from ozone generator), and 70 vol. % N₂ or 45 vol. % N₂ and 25 vol. % O₂, respectively, starting from room temperature, is brought into contact with the preheated olefin gas flow via nozzles. The reaction temperature is 300° C. After admixture, the ozone content is 0.4 vol. % and the TME content 1.62 vol. % or 1.43 vol. % (at the higher O₂ content). The O₂ content is either 8.3 vol. % or 16.7 vol. % (in case of admixture of 25 vol. % O₂ in the O₃/NOₓ gas stream). The bulk residence time in the reaction zone is 9.6 ms.

Acetone and pinacolone are found as side products.

The parameters at the working point at an O₂ content of 8.3 vol. % are:
TME conversion: 43.7%
selectivity for TME oxide: 87.4 mol. %
TME converted/O₃ employed: 1.78 (molar)
space-time-yield: 4950 g TME oxide/hr/(liter reactor volume).

The parameters at the working point at an O₂ content of 16.7 vol. % are:
TME conversion: 45.2%
selectivity for TME oxide: 89.6 mol. %
TME converted/O₃ employed: 1.64 (molar)
space-time-yield: 4650 g TME oxide/hr/(liter reactor volume).

The following terms in the examples are to be understood as:

$$Residence time = residence time of the gas mixture in the reaction zone of the flow reactor \\ \begin{matrix}olefin content in the application gas \\ \begin{matrix}ozone content in the application gas \\ [ vol . \% ]\end{matrix}\end{matrix} \} = based on the total flow in the reaction zone$$

$$conversion of olefin [ mol . \% ] = ratio of converted moles of olefin to moles of olefin employed \times 100 \%$$

$$selectivity for epoxide [ mol . \% ] = ratio of epoxide mols generated to olefin mols converted \times 100 \%$$

$$olefin converted / {O}_{3} employed ( Δ [ olefin ] / {[ {O}_{3} ]}_{0} ) = ratio of converted mols of olefin to mols of ozone employed$$

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

I claim:

1-7. (canceled)

8. A process for producing epoxides by oxidation of olefins in a homogeneous gas phase reaction, the method comprising reacting an olefin, added by use of a carrier gas, in a flow reactor with a gas mixture of ozone and NO₂ and/or NO as oxidants without use of a catalyst, mixing the ozone and NO₂ and/or NO in a mixing chamber connected upstream to the flow reactor, wherein in a reaction zone of the flow reactor the olefin is reacted at a reaction temperature of about 150° C. to about 450° C. and a pressure of 250 mbar to 10 bar with the gas mixture of the oxidant, wherein the carrier gas containing the olefin is preheated in a preheating zone of the flow reactor to a temperature of 250° C. to 650° C., and wherein the gas mixture of the oxidant from the mixing chamber, having ambient temperature, is turbulently admixed with the olefin in the reaction zone of the flow reactor, so that the reaction temperature is reached during the mixing and the ratio of olefin gas flow and gas flow of the oxidant is 5:1 to 1:1.

9. The process according to claim 8, wherein the reaction in the flow reactor is carried out at 500 to 2000 mbar.

10. The process according to claim 8, wherein the reaction in the flow reactor is carried out at more than 1000 mbar.

11. The process according to claim 8, wherein the reaction in the flow reactor is carried out at atmospheric pressure.

12. The process according to claim 8, wherein the olefin-containing carrier gas is preheated in the preheating zone of the flow reactor to a temperature of 400° C. to 550° C.

13. The process according to claim 8, wherein the reaction temperature in the reaction zone of the flow reactor is about 200° C. to about 350° C.

14. The process according to claim 8, wherein the ratio of olefin gas flow to gas flow of the oxidant is 4:1 to 2:1.

15. The process according to claim 8, wherein the carrier gas for the olefin and for the gas mixture of the oxidant comprises a gas selected from an inert gas, oxygen, air or mixtures thereof.

16. The process according to claim 8, wherein the carrier gas for the olefin and for the gas mixture comprises nitrogen.

17. The process according to claim 8, wherein the ozone is supplied to the reaction as an ozone/oxygen-mixture.
